(19) United States
(12) Patent Application Publication
(10) Pub. No.: US 2015/0182473 A1
(43) Pub. Date: Jul. 2, 2015

(54) TRANSDERMAL PATCHES WITH DISCRETE CARBON NANOTUBES

(71) Applicant: Molecular Rebar Design, LLC, Austin, TX (US)

(72) Inventors: Clive P. Bosnyak, Dripping Springs, TX (US); Kurt W. Swogger, Austin, TX (US); Nancy Henderson, Austin, TX (US); Paul Everill, Austin, TX (US)

(21) Appl. No.: 14/585,730

(22) Filed: Dec. 30, 2014

Related U.S. Application Data
(60) Provisional application No. 61/921,557, filed on Dec. 30, 2013.
(60) Provisional application No. 61/929,437, filed on Jan. 20, 2014.

(51) Int. Cl. A61K 9/70; A61N 5/06; A61N 1/30; A61K 31/455; A61M 35/00

(52) U.S. Cl. CPC A61K 9/703; A61K 31/455; A61M 35/00; A61N 1/303; A61N 5/062

(57) ABSTRACT

A transdermal patch comprising discrete open-ended carbon nanotubes is disclosed.

### CROSS REFERENCE TO RELATED APPLICATIONS

This application claims priority from U.S. Provisional Application Serial No. 61/921,557, filed Dec. 30, 2013, and U.S. Provisional Application Ser. No. 61/929,437, filed Jan. 20, 2014, the disclosures of each of which are hereby incorporated in their entirety by reference.

### STATEMENT REGARDING FEDERALLY SPONSORED RESEARCH

Not applicable

### BACKGROUND

The present disclosure relates to a transdermal patch for drug delivery using discrete carbon nanotubes and methods to obtain preferred structures.

The use of transdermal patches for the delivery of various drug systems has met with increasing success in the pharmaceutical industry, particularly in view of specific problems which have arisen in connection with drugs taken by other means, and because of their implications in terms of long term application of drugs in a particular simple manner. Descriptions of transdermal drug delivery can be found in the literature such as “Transdermal drug delivery: principles and opioid therapy.” L. Margetts and R. Sawyer, Oxford Journal, Medicine, BJA:CEACCP, Vol. 7, Issue 5, pp 171-176, 2007. The drugs can be employed in admixture with an adhesive base system for application to the skin, or non-adhesive base system having an outer drug permeable adhesive layer. The ability to do this with various types of drugs can be impeded by various considerations, such as differences in viscosity, solubility, therapeutic drug delivery rate, drug migration within the system, and the like. Although the device of the present invention has specific application in transdermal patches, the device has general application for the release of an active agent to the skin or mucosa of a host. In this regard, the device has application in active agent delivery systems which include, but are not limited to, transmucosal, buccal, sub-lingual and medicated wound care.

Simple monolithic transdermal systems incorporate their active agents, i.e., drugs, directly into a single pressure sensitive adhesive layer. These systems have the advantage of being thin, elegant, and relatively easy to manufacture, but must compromise between optimizing the adhesive matrix for drug delivery versus its ability to adhere to the skin.

The known “double-disk” transdermal patch uses a larger auxiliary patch over a smaller active agent delivery patch to improve or ensure adhesion to the skin. The adhesive matrixes of the inner and outer patches can be independently optimized for active agent delivery and adhesion, respectively. When the inner and outer patches are laminated together to form the completed system, their adhesive matrixes come into direct contact and begin to equilibrate. As the systems equilibrate, time-dependent changes occur such as the loss of active agents from the inner patch and the simultaneous accumulation of active agents in the outer patch. This phenomenon can alter the performance of the transdermal patch if any of the components in the inner patch, especially those that are needed to achieve or sustain active agent delivery, have appreciable affinity for the outer patch adhesive matrix. Moreover, this effect will become more profound with time until equilibrium is achieved.

Physical methods to increase the rate of drug delivery include the use of iontophoresis. This is the application of an electric field to drive charged particles across the skin. The charged drug is dissolved in an electrolyte solution surrounding an electrode of the same polarity and placed in contact with the skin. The opposing electrode placed elsewhere on the body completes the circuit. When an electromotive force is applied, the drug is repelled from the electrode into the skin and passes across the stratum corneum, towards the opposite electrode. The movement of charged molecules causes convective motion of the solvent, which drags neutrally charged molecules along, a process called electro-osmosis. The passage of electric current may also transiently increase the permeability of the skin. Iontophoresis can be used to deliver boluses of a drug, and has been utilized in the development of the fentanyl PCA patch. However, only small voltages can be applied and so the improvement of drug delivery rates by iontophoresis are limited.

Therefore, there remains a need to develop a transdermal patch delivery system having more control over the rate and efficiency of delivery of the drug. It is even more desirable to have such a transdermal patch that has voltage or current controlled rate of drug transport and be linked to a sensor that can monitor the drug concentration and/or controls the voltage or current that can be applied to the transdermal patch.

There is also a need for the transdermal patch to wirelessly transfer information to and from a receiving station such as a computer or cell phone. This can be achieved by embedding a chip into the transdermal patch with functions such as sensing and wireless transmission.

### SUMMARY OF THE INVENTION

The present inventors have discovered that a plurality of discrete carbon nanotubes offer novel capabilities to a transdermal patch wherein the plurality of the discrete carbon nanotubes are open-ended, at least at one end, carbon nanotubes. Preferably, the discrete carbon nanotubes are substantially non-cytotoxic.

Another embodiment of this invention is a transdermal patch comprising a plurality of discrete open-ended, at least at one end, carbon nanotubes, wherein the plurality of discrete open-ended, at least at one end, carbon nanotubes further comprise an amount of functional groups of at least about 1 percent by weight of the dry open-ended discrete carbon nanotubes. The discrete open-ended carbon tubes are dispersed within a medium fabricated into a fiber or film.

Yet another embodiment of this invention is a transdermal patch comprising a plurality of discrete open-ended carbon nanotubes, wherein the plurality of discrete open-ended carbon nanotubes are substantially cleaned of catalytic residues, i.e., preferably less than about 1% by weight, more preferably less than about 0.5% by weight, most preferably less than about 0.2% by weight and especially less than about 0.1% by weight of the tubes comprise catalytic residues. The catalytic residues are also free of Cobalt and Nickel.

In yet another embodiment of this invention is a transdermal patch comprising a plurality of discrete open-ended carbon nanotubes dispersed within a medium fabricated into a fiber or film, wherein the plurality of discrete open-ended carbon nanotubes have a length less than about 4 micrometers, preferably less than about 3 micrometers and more preferably less than about 2 micrometers. The plurality of discrete open-ended carbon nanotubes may have a length or diameter distribution modality, preferably bimodal.

An additional embodiment of this invention is a transdermal patch comprising a plurality of discrete open-ended carbon nanotubes dispersed within a medium fabricated into a fiber or film, which discrete open-ended carbon nanotubes having an aspect ratio of from about 10 to about 500, preferably about 25 to about 200 and most preferably about 50 to about 120.

A further embodiment of this invention is a transdermal patch comprising a plurality of discrete open-ended carbon nanotubes dispersed within a medium fabricated into a fiber or film, that further comprise a drug or a medicament.

In one embodiment of this invention is a transdermal patch comprising a plurality of discrete open-ended carbon nanotubes wherein the discrete carbon nanotubes are further associated with a medium, such as a polymer. The polymer is selected from a group of polymers that do not exhibit cytotoxity. The polymer may also be selected from a group of polymers that are biodegradable. The polymer comprises a weight percentage range of about 1 to about 99, preferably less than about 90 percent, more preferably less than about 50 percent and most preferably less than about 25 percent of the discrete carbon nanotubes.

In another embodiment of this invention the transdermal patch comprises material comprising a plurality of discrete open-ended carbon nanotubes dispersed within a medium fabricated into a fiber or film, wherein the discrete open-ended discrete carbon nanotubes comprises a weight percentage range of about 0.01 to about 20 percent, preferably less than about 10 percent, more preferably less than about 2 percent and most preferably less than about 1 percent of the material of the transdermal patch.

In another embodiment of this invention is a transdermal patch comprising a plurality of discrete open-ended carbon nanotubes, wherein the plurality of discrete open-ended carbon nanotubes are oriented in the direction that drug delivery is desired.

In a further embodiment of this invention is a transdermal patch comprising a plurality of discrete open-ended carbon nanotubes dispersed within a medium fabricated into a fiber or film, further comprising one or more layers wherein discrete carbon nanotubes are in at least one layer to enable a voltage to be applied across the layer containing the drug, medicant, or medicament to be delivered.

In yet another embodiment of this invention is a transdermal patch comprising a plurality of discrete open-ended carbon nanotubes dispersed within a medium fabricated into a fiber or film, wherein at least a portion of discrete nanotubes preferably has a ratio of number average value of tube contour length (TCL)):tube end to end length (TEE) of from about 1.1 to about 3, preferably from about 1.1 to about 2.8, more preferably from about 1.1 to about 2.4, most preferably from about 1.1 to about 2 and especially from about 1.2 to about 2.

In an embodiment of this invention is a transdermal patch comprising a plurality of discrete open-ended carbon nanotubes dispersed within a medium fabricated into a fiber or film, wherein the discrete carbon nanotubes comprise a mixture of discrete carbon nanotubes with different types of attached functionalities.

In another embodiment of this invention is a transdermal patch comprising a plurality of discrete open-ended carbon nanotubes dispersed within a medium fabricated into a fiber or film, further comprising a pressure-sensitive adhesive material for adhering said transdermal patch to the skin or mucosa of a host.

A different embodiment of this invention is a method to make a transdermal patch comprising a plurality of discrete open-ended carbon nanotubes comprising the steps of:
a) selecting a medium such as a polymer,
b) dispersing a plurality of discrete carbon nanotubes within the polymer,
c) fabricating the polymer discrete carbon nanotube into a fiber or film,
d) optionally melt fabricating the film or fiber, and
e) optionally using one or more multilayer coextrusion generators.

The method can also comprise joining one or more layers of polymers. The plurality of discrete open-ended carbon nanotubes can also be deposited as a layer or on a layer surface by one or more deposition methods comprising spraying, inkjet printing, transfer printing, lamination, painting or spin coating.

Another embodiment of this invention is a method to make a transdermal patch comprising a plurality of discrete open-ended carbon nanotubes comprising the steps of:
a) selecting a material medium,
b) selecting a medicament,
c) dispersing a plurality of discrete carbon nanotubes within the medium and medicament,
d) fabricating the medium, medicament and discrete carbon nanotube into a fiber or film, and
e) optionally using one or more multilayer coextrusion generators. Fabricating the fiber or film in step (d) can be a melt fabricating step.

Yet another invention embodiments is a conductive transdermal patch layer comprising a medicament in ionic form and from about 0.01 to about 1 weight % of a plurality of discrete carbon nanotubes, wherein the patch has a conductivity of at least 50% greater than, and up to 500% greater than, a transdermal patch consisting essentially of the same about amount of a plurality of discrete carbon nanotubes and an absence of the medicament in ionic form, preferably wherein the conductive transdermal patch comprises a medicament in ionic form and from about 0.25 to about 0.75% weight of a plurality of discrete carbon nanotubes, wherein the patch has a conductivity of at least 50% greater than, and up to 200% greater than, a transdermal patch consisting essentially of the same about amount of a plurality of discrete carbon nanotubes and an absence of the medicament.

Another invention embodiment is a transdermal patch comprising a plurality of discrete carbon nanotubes dispersed within a medium fabricated into a fiber or film, wherein a plurality of discrete carbon nanotubes are open-ended at least at one end.

### BRIEF DESCRIPTION OF THE DRAWINGS

FIG. 1 shows normalized current versus time (seconds) plotted graphically for Example 1 and Control 1 described in the present invention.

### DETAILED DESCRIPTION OF THE PREFERRED EMBODIMENTS

In the following description, certain details are set forth such as specific quantities, sizes, etc. so as to provide a thorough understanding of the present embodiments disclosed herein. However, it will be evident to those of ordinary skill in the art that the present disclosure may be practiced without such specific details. In many cases, details concerning such considerations and the like have been omitted inasmuch as such details are not necessary to obtain a complete understanding of the present disclosure and are within the skills of persons of ordinary skill in the relevant art.

While most of the terms used herein will be recognizable to those of ordinary skill in the art, it should be understood, however, that when not explicitly defined, terms should be interpreted as adopting a meaning presently accepted by those of ordinary skill in the art. In cases where the construction of a term would render it meaningless or essentially meaningless, the definition should be taken from Webster's Dictionary, 3rd Edition, 2009.

The main components to a transdermal patch are, but not limited to, a) a liner that protects the patch during storage which is removed prior to use, b) a drug or combinations of drugs that can be in the form of a solution or gel in direct contact with release liner, c) an adhesive that serves to adhere the components of the patch together along with adhering the patch to the skin, d) a membrane which controls the release of the drug from the reservoir and multi-layer patches, and e) a backing that protects the patch from the outer environment.

A type of transdermal patch can be, for example, a single-layer drug-in-adhesive where the adhesive layer of this system also contains the drug. In this type of patch the adhesive layer not only serves to adhere the various layers together, along with the entire system to the skin, but is also responsible for the releasing of the drug. The adhesive layer is surrounded by a temporary liner and a backing.

Another type of transdermal patch can be a Multi-layer drug-in-adhesive where two or more adhesive layers are responsible for the releasing of the drug, sometimes together with a membrane layer. One of the layers is for immediate release of the drug and other layer or layers are for control release of drug from the reservoir. This patch also has a temporary liner-layer and a permanent backing. The rate of drug release from this type of transdermal patch depends on permeability and diffusion of drug molecules through the membrane and adhesive layers.

Yet another type of transdermal patch is a Reservoir transdermal patch. Unlike the single-layer and multi-layer drug-in-adhesive systems the reservoir transdermal system has a compartment that contains a drug solution or suspension or gel separated by the adhesive layer. This patch is also backed by the backing layer. In this type of system the rate of release is essentially zero order. A zero-order rate of release is a release that proceeds at a rate that is independent of the drug concentration. Here the drug reservoir is encapsulated in a shallow compartment usually molded from drug impermeable metallic plastic laminates, with one side made of a rate controlling membrane made up of polymeric membrane, such as, but not limited, to poly(ethyl vinyl acetate), (also known as EVA) which is the copolymer of ethylene and vinyl acetate. The weight percent vinyl acetate usually varies from 10 to 40%, with the remainder being ethylene.

Another type of transdermal patch is a Matrix transdermal patch. The Matrix system has a drug layer of a semisolid matrix containing a drug solution or suspension or gel. The adhesive layer in this patch surrounds the drug layer partially overlaying it. It is also known as a monolithic device.

A discrete carbon nanotube, DCNT, serving as a component of a transdermal patch used in this invention can consist of single wall, double wall or multiwall graphene shells.

The term “Discrete Carbon Nanotubes (DCNT)” means carbon nanotubes that are unbundled or untangled, not tangled as a mass, from their state as made in the reactor with catalysis and do not require further cutting of their length to be able to be substantially separated from one another along the length of the carbon nanotube. The term “plurality” means more than 50 percent by weight. The discrete carbon nanotubes can be individually dispersed in a given medium by selection of the thermodynamic interaction of the tube surface and the medium and the tube concentration. For example, discrete carbon nanotubes of this invention can be dispersed easily at a concentration of 1% by weight in water at room temperature using 0.5% by weight of polyvinyl alcohol or polyvinylpyrrolidone of molecular weight about 50,000 Daltons and a sonicator or high shear mixer.

During the process of making discrete or exfoliated carbon nanotubes (which can be single, double and multiwall configurations), from bundles or entangled masses of carbon nanotubes, the nanotubes are cut into segments with at least one open end and residual catalyst particles that are exterior or interior to the carbon nanotubes as received from the manufacturer are removed. This cutting of the tubes helps with exfoliation. The cutting of the tubes reduces the length of the tubes into carbon nanotube segments that are defined here as Molecular Rebar. Proper selection of the carbon nanotube feed stock related to catalyst particle type and distribution in the carbon nanotubes allows more control over the resulting individual tube lengths and overall tube length distribution. A preferred selection is where the internal catalyst sites are evenly spaced and where the catalyst is most efficient. A further preferred selection is where there are Stone-Wales defects present along the wall or walls of the carbon nanotube. Individual discrete carbon nanotubes can have an aspect ratio of from about 10 to about 500, preferably 25-200 and most preferably 50-120 for a balance of fluid viscosity, ease of discrete carbon nanotube dispersion and performance. The selection of tubes for the scaffold can be evaluated using electron microscopy and determination of the discrete or exfoliated tube distribution. In some cases it may be preferred to have a modality of aspect ratios. For example, but not limited to, a bimodal distribution of lengths to provide a range of drug elution rates and a balance of viscosity with say electrical conductivity or strength.

The bundled or entangled carbon nanotubes can be made from any known means such as, for example, chemical vapor deposition, laser ablation, and high pressure carbon monoxide synthesis. It is preferred that the carbon nanotubes are made via catalysts that are non-toxic, for example iron. The bundled carbon nanotubes can be present in a variety of forms including, for example, soot, powder, fibers, and bucky paper. Furthermore, the bundled carbon nanotubes may be of any length, diameter, or chirality. Carbon nanotubes may be metallic, semi-metallic, semi-conducting, or non-metallic based on their chirality and number of walls.

Discrete oxidized carbon nanotubes, alternatively termed exfoliated carbon nanotubes, are obtained from as-made bundled or entangled carbon nanotubes by such methods involving oxidation, such as using a combination of concentrated sulfuric and nitric acids, ozone or peroxides such as hydrogen peroxide and a high energy source which discretizes the tubes such as a sonicator. Select conditions of carbon nanotube concentrations, usually less than about 1.5% by weight of the acid, temperatures such as between about 25 and about 90 centigrade, mixing energies around about 20 to 40 megajoules per kilogram of carbon nanotubes and times of about 2 to 5 hours are desired for removal of greater than about 90% of the initial entangled clusters or bundles of carbon nanotubes. The discrete oxidized carbon nanotubes may include, for example, single-wall, double-wall carbon nanotubes, or multi-wall carbon nanotubes and combinations thereof. One of ordinary skill in the art will recognize that many of the specific aspects of this invention illustrated utilizing a particular type of carbon nanotube may be practiced equivalently within the spirit and scope of the disclosure utilizing other types of carbon nanotubes.

A preferred selection of carbon nanotubes of this invention is the incorporation of a portion of structures called Stone-Wales defects which are the rearrangement of the six-membered rings of graphene into heptagon-pentagon pairs that fit within the hexagonal lattice of fused benzene rings constituting a wall of the carbon nanotubes. These Stone-Wales defects are useful to create sites of higher bond-strain energy for more facile reaction such as oxidation of the graphene or carbon nanotube wall. These defects and other types of fused ring structures may also facilitate bending or curling along the length of the carbon nanotubes which is advantageous for maintaining fluidity of mixtures of discrete carbon nanotubes with fluids at higher concentrations of discrete carbon nanotubes. The Stone-Wales defects can be determined using resonant-vibrational spectroscopy techniques. A preferred portion of Stones-Wales defects relative to the hexagonal six-membered rings of the carbon is from about 1 to about 15%, more preferably about 2 to about 10% and most preferably about 5 to about 9%.

Stone-Wales defects are thought to be more prevalent at the end caps that allow higher degrees of curvature of the walls of carbon nanotubes. During oxidation the ends of the carbon nanotubes can be opened and also result in higher degrees of oxidation than along the walls. The discrete carbon nanotubes of this invention have a plurality of carbon nanotubes that are open at least at one end. The higher degree of oxidation and hence higher polarity or hydrogen bonding at the ends of the tubes are useful to help decrease the average contour length to end to end ratio when the tubes are present in polar media and can be used to attach molecules which could control the rate of release of a drug medicament from within the tube.

According to a preferred embodiment, the discrete carbon nanotubes are functionalized. The functional groups linked to discrete carbon nanotubes, DCNT, include, but not limited to, hydroxyl, thiol, amide, amine and carboxyl groups. The functionalized DCNT allows for the decrease in aggregation between DCNT molecules in a medium and greater affinity of stem cells and other biological moieties. The functionalization allows for interaction with inorganic metals, inorganic salts, organic molecules such as, but not limited to, polyethylene glycol, and biological species such as, but not limited to DNA, RNA, peptides, proteins and enzymes. Functionalized carbon nanotubes of the present disclosure generally refer to the chemical modification of any of the carbon nanotube types described hereinabove. Such modifications can involve the nanotube ends, sidewalls, or both. Chemical modifications may include, but are not limited to covalent bonding, ionic bonding, chemisorption, intercalation, surfactant interactions, polymer wrapping, cutting, solvation, and combinations thereof.

The transdermal patch may comprise discrete carbon nanotubes of this invention in at least one of the components of the transdermal patch. The transdermal patch may comprise discrete carbon nanotubes, wherein at least a portion of discrete nanotubes preferably has a ratio of number average value of tube contour length (TCL)):tube end to end length (TEE) of from about 1.1 to about 3, preferably from about 1.1 to about 2.8, more preferably from about 1.1 to about 2.4, most preferably from about 1.1 to about 2 and especially form about 1.2 to about 2. The values of TCL and TEE can be measured by scanning electron microscopy. The tube contour length (TCL)):tube end to end length (TEE) ratio being greater than about 1.1 aids in reducing the viscosity of the mixture of discrete carbon nanotubes and a medium such as a polymer and facilitates fabrication into fiber or film form.

The ratio of the TCL to TEE can be advantageously controlled by the degree of thermodynamic interaction between the tube surfaces and the medium. Surfactants can be usefully employed also to modify the thermodynamic interactions between the tubes and the medium of choice. Alternate means to influence the ratio of discrete carbon nanotube contour length to end to end ratio include the use of inorganic or ionic salts such as sodium chloride and organic containing functional groups such as polyethylene oxide or polyvinyl alcohol that can be attached to or contacted with the tube surfaces.

Yet another method for controlling the TCL to TEE ratio is by orientation. The discrete carbon nanotubes can be oriented by such means, although not limited, by extrusion through a circular or slit die. The orientation is facilitated by the presence of a polymer in the fluid. An example of this is employing discrete carbon nanotubes of this invention in the presence of polyvinyl alcohol and water such that polyvinyl alcohol-oriented discrete carbon nanotube fibers can be obtained by electrospinning or via orifices in the wall of a spinning centrifuge. The fibers can be stacked together, fused and cut to produce films with discrete carbon nanotubes oriented normal to the plane of the cut. Another example is where the mixture of discrete carbon nanotubes is extruded using multilayer generators as described, for example by Schrenk in U.S. Pat. No. 3,884,606, issued May 20, 1975. The multilayer generators can be configured so that stacks of layers can be obtained with the preferred orientation of the mixture of discrete carbon nanotube mixtures normal to the plane of the film.

The discrete carbon nanotubes used in the transdermal patch invention described herein need not comprise 100% discrete carbon nanotubes. In one case, some tube bundles may still exist as entangled, non-discrete tubes. However, the discrete carbon nanotubes of this invention preferably comprise of at least 50% by weight of all carbon nanotubes, more preferably greater than 75%, most preferably greater than 90% and especially 95% or more of all carbon nanotubes present. In another case other electron conductive entities may also be present. These conductive additives include, but are not limited to, other forms of carbonaceous materials such as carbon black, graphite, carbon fibers with diameters greater than 100 nanometers and graphene or graphene oxide. However, the discrete carbon nanotubes of this invention preferably comprise of at least 50% by weight of all electron conducting species, more preferably greater than 75%, most preferably greater than 90% and especially 95% or more of all electron conducting species present.

The carbon nanotubes used in this transdeinial patch invention can comprise a layer of at least 1% by weight of the layer of discrete carbon nanotubes, more preferably greater than 10%, most preferably greater than 15% and especially 25% or more discrete carbon nanotubes. The discrete carbon nanotubes with at least one open end of this invention can be used as a vesicle for the drug or combinations of drugs.

The transdermal patch comprising discrete carbon nanotubes can further comprise a medium such as polymers that are substantially non-cytotoxic. The medium can be a natural, synthetic, or semi-synthetic fiber, film, sheet, or fabric. The polymers are selected from a variety of natural, synthetic, semi-synthetic, and biosynthetic polymers that can further be biocompatible or biodegradable. A polymer based on a C—C backbone tends to resist degradation, whereas heteroatom-containing polymer backbones confer biodegradability known in the art. Biodegradability can, therefore, be engineered into polymers by the judicious addition of chemical linkages such as anhydride, ester, or amide bonds, among others. The usual mechanism for degradation is by hydrolysis or enzymatic cleavage of the labile heteroatom bonds, resulting in a scission of the polymer backbone. Macro organisms can eat and, sometimes, digest polymers, and also initiate a mechanical, chemical, or enzymatic aging. Biodegradable polymers with hydrolysable chemical bonds are researched extensively for biomedical, pharmaceutical, agricultural, and packaging applications. In order to be used in medical devices and controlled-drug-release applications, the biodegradable polymer must be biocompatible and meet other criteria to be qualified as biomaterial-processable, sterilizable, and capable of controlled stability or degradation in response to biological conditions. The chemical nature of the degradation products, rather than of the polymer itself, often critically influences biocompatibility. Poly(esters) based on polylactide (PLA), polyglycolide (PGA), polycaprolactone (PCL), and their copolymers have been extensively employed as biomaterials. Degradation of these materials yields the corresponding hydroxy acids, making them safe for in vivo use. Other bio- and environmentally degradable polymers include, but not limited to, polyvinyl alcohols, poly(hydroxyalkanoate)s such as the polyhydroxybutyrate-polyhydroxyvalerate class, polyvinylpyrrolidone, additional poly(ester)s, and natural polymers, particularly, modified poly(saccharide)s, e.g., starch, cellulose, and chitosan.

The transdermal patch comprising discrete carbon nanotubes can comprise adhesive. Preferred adhesive matrix materials for transdermal patches are polymers comprising poly(meth)acrylate, polyvinylpyrrolidone, ethylcellulose, hydroxypropylcellulose, hydroxypropylmethylcellulosephthalate, polyvinylalcohol or copolymers thereof with vinyllaurate or maleic acid, vinylacetate or copolymers thereof with vinyllaurate or maleic acid, polyvinylether butylrubber and polycaprolactam. The discrete carbon nanotubes can be incorporated into these polymers using slurry, solution or melt extrusion methods.

The transdermal patch comprising discrete carbon nanotubes can comprise discrete carbon nanotubes wherein the lengths of the discrete carbon nanotubes can be a unimodal distribution, or a multimodal distribution (such as a bimodal distribution). The multimodal distributions can have evenly distributed ranges of lengths (such as 50% of one length range and about 50% of another length range). The distributions can also be asymmetrical—meaning that a relatively small percent of discrete nanotubes can have a specific length while a greater amount can comprise another length.

The transdermal patch comprising discrete carbon nanotubes can further comprise discrete carbon nanotubes wherein the diameters of the discrete carbon nanotubes can be a unimodal distribution, or a multimodal distribution (such as a bimodal distribution). The multimodal distributions can have evenly distributed ranges of diameters (such as 50% of one diameter range and about 50% of another diameter range). The distributions can also be asymmetrical—meaning that a relatively small percent of discrete nanotubes can have a specific diameter while a greater amount can comprise another diameter. The distribution of diameters and/lengths can be used to control the rate of diffusion of the drug through or from the interior of open-ended discrete carbon nanotubes.

The transdermal patch can comprise discrete carbon nanotubes wherein the discrete carbon nanotubes can comprise combinations of functionality. An example is a portion of discrete carbon nanotubes having 2% by weight of carboxylic acid groups are admixed with a portion of discrete carbon nanotubes having 2% by weight amine groups for the purpose of, but not limited to, combining different medicaments or altering the rate of delivery of the medicament.

Other aspects of the invention involving discrete carbon nanotubes in a transdermal patch device include a controlled drug delivery system comprising Molecular Rebar, MR, or DCNT and at least one active drug ingredient, preferably wherein the active drug ingredient is substantially within the volume of the MR or DCNT, or closely associated with the surfaces of the carbon nanotube, either on the innermost or outermost graphene layer. The controlled drug delivery method can be activated by radiation such as electro-magnetic radiation, heat or ultraviolet radiation (e.g., magnetic resonance imaging, MRI).

It is possible to use active substances which can be applied in transdermal manner and typical examples of these are given below.

Nicotine.

Corticosteroids: hydrocortisone, prednisolone, beclomethasone-propionate, flumethasone, triamcinolone, triamcinolone-acetonide, fluocinolon, fluocinolinacetonide, fluocinolon-acetonide acetate, clobetasolpropionate, etc.

Analgesics, anti-inflammatory agents: acetaminophen, mefenamic acid, flufenamic acid, diclofenac, diclofenacsodium-alclofenac, oxyphenbutazone, phenylbutazone, ibuprofen, flurbiprofen, salicylic acid, 1-menthol, camphor, sulindac-tolmetin-sodium, naproxen, fenbufen, etc.

Hypnotically active sedatives: Phenobarbital, amobarbital, cyclobarbital, triazolam, nitrazepam, lorazepam, haloperidol, etc.

Tranquilizers: fluphenazine, thioridazine, lorazepam, flunitrazepam, chloropromazine, etc.

Antihypertensives: pindolol, indenolol, nifedipin, lofexidin, nipradinol, bucumolol, etc.

Antihypertensively acting diuretics: hydrothiazide, bendroflumethiazide, cyclopenthiazide, etc.

Antibiotics: penicillin, tetracycline, oxytetracycline, fradiomycin sulfate, erythromycin, chloramphenicol, etc.

Anesthetics: lidocane, benzocaine, ethylaminobenzoate, etc.

Antimicrobiological agents: benzalkonium chloride, nitrofurazone, nystatin, acetosulfamine, clotrimazole, etc.

Antifungal agents: pentamycin, amphotericin B, pyrrolnitrin, clotrimazole, etc.

Vitamins: vitamin A, ergocalciferol, chlolecalciferol, octotiamine, riboflavin butyrate, etc.

Antiepileptics: nitrazepam, meprobamate, clonazepam, etc.

Coronary vasodilators: dipyridamole, erythritol tetranitrate, pentaerythritol tetranitrate, propatylnitrate, etc.

Antihistamines: diphenyl hydramine hydrochloride, chlorpheniramine, diphenylimidazole, etc.

Antitussives: dertromethorphan (hydrobromide), terbutaline (sulphate), ephedrine (hydrochloride), salbutamol (sulphate), isoproterenol (sulfate, hydrochloride), etc.

Sexual hormones: progesterone, etc.

Thymoleptics: doxepin, etc.

Further medicaments/pharmaceuticals: 5-fluorouracil, fentanyl, desmopressin, domperdon, scopolamine (hydrobromide), peptide, insulin, combretastatins, etc.

Obviously, this list is not exhaustive.

Most drugs are weak electrolytes. Weak acids and weak bases undergo ionisation in solution. Drugs are more soluble in water when they are in ionised form. Unionised drugs are usually poorly water soluble. The extent of ionisation of drug in a solution depends on the dissociation constant and the pH of the medium. For example, alkaloidal salts are more soluble in acidic pH and begin to precipitate as the pH increases. On the other hand, phenobarbitone is more soluble in alkaline Ph and begins to precipitate as the pH decreases.

In the transdermal patch compositions of this invention, a pharmaceutically acceptable carrier for the drug may be conventional for formulation, including carbohydrates (e.g., lactose, amylose, dextrose, sucrose, sorbitol, mannitol, starch, cellulose), gum acacia, calcium phosphate, alginate, gelatin, calcium silicate, microcrystalline cellulose, polyvinylpyrrolidone, water, salt solutions, alcohols, gum arabic, syrup, vegetable oils (e.g., corn oil, cotton-seed oil, peanut oil, olive oil, coconut oil), polyethylene glycols, methyl cellulose, methylhydroxy benzoate, propylhydroxy benzoate, talc, magnesium stearate and mineral oil, but not limited to the pharmaceutical compositions of this invention, further may contain wetting agent, lubricant, stabilizer, or mixtures of these substances. In some cases surfactants may be used to aid in the dispersion of drugs. Examples of surfactants, but not limiting, are cationic, like cetyltrimethylammonium bromide, anionic like sodium dodecylsulphonate, or nonionic like polyethyleneglycolalkylethers. Details of suitable pharmaceutically acceptable carriers and formulations can be found in Remington's Pharmaceutical Sciences (19th ed., 1995), which is incorporated herein by reference.

The correct dosage of the pharmaceutical compositions of this invention will be varied according to the particular formulation, the mode of application, age, body weight, and gender of the patient, diet, time of administration, route of administration, condition of the patient, excretion rate, and reaction sensitivity, and so on.

According to conventional techniques known to those skilled in the art, the discrete carbon nanotube compositions of this invention can be formulated with pharmaceutical acceptable carrier and/or vehicle, finally providing several forms including a unit dosage form or a multi-unit dosage forms. The dosage forms can comprise a solution, a suspension or an emulsion in an oily or aqueous medium as well as further dispersions or stabilizers.

The film, fiber or foam structures comprising discrete carbon nanotubes may also comprise layers differing in composition. For example, in a tape the layers may consist of different amounts of discrete carbon nanotubes, different types of discrete carbon nanotubes, or different additives or concentration of additives.

### General Process to Make Discrete Carbon Nanotubes (DCNT) or Molecular Rebar (MR)

As manufactured carbon nanotubes in the form of fibrous bundles or granules can be obtained from different sources to make discrete carbon nanotubes. However, for the examples used herein, entangled carbon nanotubes obtained from CNano, grade Flotube 9000 are used. Flotube 9000 carbon nanotubes have less than 5% by weight of impurities of which about 4% by weight or less are residual catalyst. The catalyst residues are determined by energy dispersive X-ray to comprise Iron and Aluminum. The average number of walls which make up the carbon nanotube is about 10. The tube diameter average is about 13 nm (a later table herein lists other tube diameters of about 12.5 nm) as determined from several tube diameters measured by scanning electron microscopy (SEM). Carbon nanotube manufacturers can have higher percentage impurities and much broader and larger diameter tube distributions depending on manufacturing technique. The carbon nanotube diameter and diameter distributions are determined by and characteristic of the process and catalyst conditions used to make the carbon nanotubes. Other carbon nanotube manufacturers include Arkema and Southwest Nanotechnologies.

The resulting discrete carbon nanotube length and length distributions from the process of making MR are related in part to the initial catalyst efficiency and process conditions for the entangled or bundled carbon nanotubes. The MR process cuts the entangled or bundled carbon nanotubes preferentially at a catalyst site, and/or large concentration of Stone-Wales defects. The larger diameter carbon nanotubes with larger number of walls are generally more difficult to cut than smaller diameter carbon nanotubes and so longer discrete carbon nanotubes resulting from the MR process tend to have larger diameters.

### Method to Make Discrete CNT

Nitric acid solution (greater than about 60 weight % concentration, preferably above 65% nitric acid concentration, in water) in conjunction with a controlled high energy dispersive mixer is used to exfoliate the carbon nanotubes. Alternate oxidation methods such as air at 350° C., mixed acid systems (e.g., nitric and sulfuric), peroxides such as hydrogen peroxide or persulfates such as sodium persulfate, and permanganates, such as potassium permanganate can be used.

One illustrative process for making discrete carbon nanotubes follows: A 16 liter mixture of 1.2% by weight of CNT's (obtained from CNano, grade Flotube 9000) in >65% nitric acid, is pumped at 1.5 l/min. thru a 1000 watt Heilsher cell using a 34 mm diameter sonitrode. The back pressure is 30 psi, the amplitude is set at 85% and the recorded watts are at 500-600. After all of the 16 liters are pumped through the cell, the CNT slurry is drained back and the process is repeated until the CNT's are exfoliated to the desired specification, for example as tested by optical microscopy and/or UV absorption. The tubes are washed with distilled water to pH greater than 3 and dried using convective air at 120° C.

The degree of oxidation can be measured by several tests such as O1s spectroscopy, energy dispersive X-ray and thermo-gravimetric analysis. Quantification of carboxylic and hydroxyl groups can be determined by titration using a sodium hydroxide solution.

An example of measurements of the lengths of discrete carbon nanotubes made by varying the degree of oxidation and intensity of mixing is given in Table 1

TABLE 1
Lengths (nm)

| | Condition 1 | Condition 2 | Condition 3 |
|---|---|---|---|
| Mean | 424 | 487 | 721 |
| Standard Error | 25.3 | 34.9 | 50 |
| Median | 407 | 417.0 | 672 |
| Standard Deviation | 177 | 281 | 315 |
| Sample Variance | 31461 | 79108 | 99418 |
| Kurtosis | −0.83 | 1.5 | −0.02 |
| Skewness | 0.03 | 1.2 | 0.64 |
| Range | 650 | 1270.0 | 1364 |
| Minimum | 85 | 85.0 | 161 |
| Maximum | 735 | 1355 | 1525 |

Condition 1 is an example of a narrow distribution with low mean length. Condition 2 is an example of broad distribution with low mean length. Condition 3 is an example of high mean length and broad distribution.

To determine tube lengths, a sample of tubes is diluted in isopropyl alcohol and sonicated for 30 minutes. It is then deposited onto a silica wafer and images are taken at 15 kV and 20,000× magnification by SEM. Three images are taken at different locations. Utilizing the JEOL software (included with the SEM) a minimum of 2 lines are drawn across on each image and measure the length of tubes that intersect this line.

Skewness is a measure of the asymmetry of a probability distribution. A positive value means the tail on the right side of the distribution histogram is longer than the left side and vice versa. Positive skewness is preferred which indicates means more tubes of long lengths. A value of zero means a relatively even distribution on both sides of the mean value. Kurtosis is the measure of the shape of the distribution curve and is generally relative to a normal distribution. Both skewness and kurtosis are unitless.

The following table shows representative values of discrete carbon nanotubes diameters:

TABLE 2
Diameter (unrelated to condition above)

| | |
|---|---|
| Mean diameter (nm*) | 12.5 |
| Median diameter (nm) | 11.5 |
| Kurtosis | 3.6 |
| Skewness | 1.8 |

Calculated tube contour length aspect ratio for conditions 1, 2 and 3 are 34, 39, and 58, respectively.
*nm = nanometer

### Method to Make a Layer of Discrete Carbon Nanotubes by Spraying

Discrete open-ended carbon nanotubes (1 gram) having a length distribution of composition 3 and 2% by weight of oxidized species is admixed with a solution of water (98 grams) and 1 gram of polyvinyl alcohol (Aldrich, 88% hydrolyzed, molecular weight 31-70 KDa) at room temperature. The pH of the mixture is adjusted to about pH 7 using sodium hydroxide. The mixture is sonicated in a sonicator bath for 30 minutes. The mixture is sprayed onto a glass plate using an airbrush at 50 psi pressure to give a uniform coating of the discrete carbon nanotubes and polyvinylalcohol and the coating dried in air. The film thickness can be varied by repeating the spraying and drying process. The film is removed from the glass surface and can be cut to a desired shape. The surface conductivity of the coating after drying, measured using a digital multimeter, is 4×10² ohms-square. The layer can be used as an electrode in a transdermal patch or imbibed with a solvent such as water or a solution of a drug as a component of a transdermal patch.

### A Method to Include a Medicament

Discrete carbon nanotubes with a portion of open ends are washed to pH 6 and dried in vacuo for 4 hours at 80 degrees centigrade. (1 g) of the dried discrete carbon nanotubes are added to anhydrous ethanol (100 g) containing 0.05 g of retinoic acid (also known as vitamin A acid). The mixture is sonicated for 30 minutes at 50 degrees centigrade then cooled to room temperature. The discrete carbon nanotubes with adsorbed retinoic acid are filtered and dried. The ethanol may optionally be removed via a vacuum. 1 gram of the discrete carbon nanotubes with retinoic acid are mixed with 98 grams of water at pH about 7 and 1 gram of polyvinyl alcohol (Aldrich, 88% hydrolyzed, molecular weight 31-70 KDa) at room temperature and sonicated in a water bath for 15 minutes to obtain a stable dispersion of discrete carbon nanotubes with retinoic acid.

### A Method to Orient the Discrete Carbon Nanotubes and Include a Medicament

48.45 g of polyethylene oxide of molecular weight 300,000 daltons (Alfa Aesar and 8.55 g of dry discrete carbon nanotubes of length distribution of composition 3 were weighed in a paper cup and mixed with a spoon thoroughly. A Haake mixer with Roller-Rotor configuration mixing blades was pre-heated to 90° C. and rpm was set to 20. The mixture was added slowly into the cavity using the hopper over a period of four minutes. Thereafter the rpm was raised to 80 and mixing continued for 30 minutes. The final melt temperature was 150° C. and the mixture removed.

The cooled mixture can be cut into small pieces and extruded using a Micro 15 Twin-Screw extruder (DSM Research Netherlands) and a filament die. The mixture of discrete carbon nanotubes can be extruded at 120° C. and the oriented filament wound over a bobbin such that the filament fuse together and form a tube. The fused filaments are removed from the bobbin and the tube sliced along the length of the tube to the desired thickness to form a layer wherein the majority of the tubes are oriented approximately in the direction of the film thickness. This layer can be used as a membrane layer or as a reservoir for drugs in a transdermal patch configuration.

A polyethylene oxide layer of about 200 micrometers thick containing 15% by weight of discrete carbon nanotubes as described previously is imbibed with an ethyl acetate solution of 5% by weight of a eutectic mixture of ibuprofen (IBP) with Shea butter (melting point about 30 centigrade), ratio by weight 30 to 70 of IBP to Shea butter, respectively. The composition ratios of other drug/pro-drug combinations can be tuned using a differential scanning calorimeter so as to produce a eutectic point close to that of the skin temperature i.e. 32-34° C. The ethyl acetate is removed from the layer in vacuo at room temperature. The rate of drug delivery can be increased by microwave, infra-red or radiofrequency radiation which is absorbed by the carbon nanotubes, thereby causing a heating effect. The dielectric constant of the layer or the electrical conductivity can be determined to monitor the rate of drug release.

### A Method to Control the Rate of Drug Delivery Using Voltage

A polyethylene oxide layer of about 200 micrometers thick containing 15% by weight of discrete carbon nanotubes, as described previously, is imbibed with an aqueous solution of Fentanyl HCL (molecular weight 286 g mol-1.) Sodium chloride is added as an electrolyte. Approximately 4 milligrams of Fentanyl per 100 cm2 of patch is employed. The drug diffusion kinetics is analyzed by using a Franz Diffusion cell that measures the drug flux across an “artificial skin” silastic membrane maintained at skin temperature and where an anode grid wire electrode is in contact with the layer containing discrete carbon nanotubes and a cathode grid wire electrode is in contact with the synthetic skin. The drug content concentration is measured using HPLC. On applying current 170 microamps across the membrane an increase in the rate of drug delivery is observed relative to when no voltage is applied.

A control made and tested as above but without discrete carbon nanotubes shows less rate of drug delivery than the system with discrete carbon nanotubes.

In the following Examples is demonstrated the effect of addition of discrete carbon nanotubes on the conductivity of a films containing sodium nicotinate, and the rate of ion transport across a cathode and anode electrode with an applied constant voltage.

5 g of Nicotinic acid is added to 90 g of water in a glass beaker at room temperature with a pH probe and a stirred with magnetic stirrer. Drops of 25% sodium hydroxide in water are added until a pH of 8 was obtained. At pH 6 the nicotinic acid is observed to be fully dissolved. Sufficient additional water is added to make a 5% w/w aqueous solution of the sodium nicotinate.

A stock solution of 1% w/w dispersed discrete functionalized carbon nanotubes in water with average length 850 nm and average diameter 12.5 nm, oxidation level 2.5% wt, is prepared by first dissolving 0.05 g of polyvinylpyrrolidone (40,000 Da, Sigma-Aldrich) at room temperature in 9.85 g of water at pH 8. 0.1 g of dry discrete carbon nanotubes is then added and the mixture sonicated in a water bath (Branson Sonicator bath) for 30 minutes at room temperature. A stable dispersion of discrete carbon nanotubes is obtained.

### EXAMPLE 1

0.93 g of polyvinylpyrollidone 40,000 Da (Sigma Aldrich) and 0.93 g polyethylene glycol 200 Da, (Sigma-Aldrich) is added to a glass bottle. 16 g of water at pH 8 (using sodium hydroxide) is added and 2 g of the 5% by weight sodium nicotinate solution. The mixture is dissolved with shaking over 10 minutes. Then 1 g of the 1% discrete carbon nanotube solution is added and the entire mixture stirred thoroughly with a spatula. The mixture is then poured into a petrie dish and dried in an air oven for 12 hours. A viscous black liquid is obtained with 5% wt. sodium nicotinate and 0.5% wt. discrete carbon nanotubes.

### EXAMPLE 2

The same composition as example 1 but with no sodium nicotinate.

### Control 1

The same composition as example 1 but with no addition of discrete carbon nanotubes. A clear viscous liquid is obtained after drying.

### Control 2

The same composition as example 1 but with no addition of sodium nicotinate or discrete carbon nanotubes. A clear viscous liquid is obtained after drying.

The conductivity of the viscous liquids are determined using a cell containing two copper electrodes of diameter 1.9 cm. The electrodes were positioned 1.3 mm apart. An LCR meter (Agilant 4263B) is used to measure the impedance bulk conductivity at 25 degrees centigrade at 1 Khz and level 1 volt. The units of conductivity are Siemen/cm.

| | | | Bulk | |
|---|---|---|---|---|
| | % | % Sodium | Conductivity | |
| | DCNT | Nicotinate | S/cm | Range |
| Example 1 | 0.5 | 5 | 4.29E−04 | 4.21E−04 |
| Control 1 | 0 | 5 | 1.55E−04 | 1.49E−04 |
| Example 2 | 0.5 | 0 | 8.44E−06 | |
| Control 2 | 0 | 0 | 5.99E−06 | |

Comparison of Example 1 with Control 1 and Example 2 with Control 2 shows the improvement of conductivity of a transdermal patch layer containing sodium nicotinate with 0.5% addition of discrete carbon nanotubes. The improved conductivity is important for improvements in the efficiency of iontophoresis. The difference in the conductivity measurements between Example 1 and Example 2 (4.21 e-4 S/cm) and Control 1 and Control 2 (1.49 e-4 S/cm) represents the improved range or sensitivity of conductivity of the sodium nicotinate with addition of discrete carbon nanotubes of this invention, a factor of 2.8×.

The following example illustrates the improvement in ion transport of drugs under a voltage gradient in the presence of discrete carbon nanotubes of this invention.

Using Example 1 and Control 1, the viscous liquids are packed between the electrode cell and subjected to a constant voltage of 4 volts DC (30.8 V/cm) for at least 75 minutes at room temperature. The electrodes are shorted for 5 seconds to reduce capacitance build-up, then the polarity is reversed and the current determined as a function of time for up to 15 minutes. The rate of current drop is taken as being proportional to the mobility of the ions through the viscous medium under the voltage gradient.

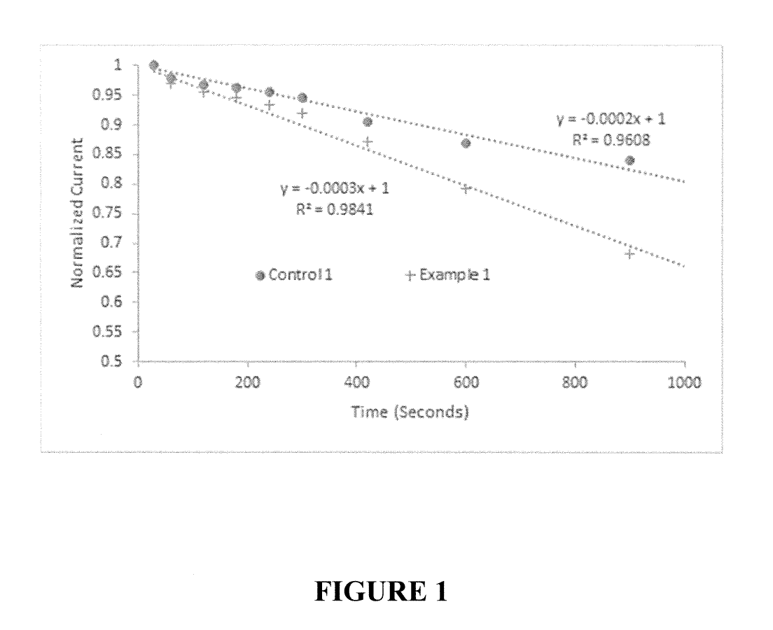

Shown in FIG. 1 is a plot of the current normalized by the current after 0.5 minutes versus time (seconds) for Example 1 and Control 1.

FIG. 1 shows that the mobility of ions of Example 1 in the presence of 0.5% wt. discrete carbon nanotubes with a plurality of open ends is about 50% greater than Control 1 with the same concentration of sodium nicotinate, but without discrete carbon nanotubes of this invention.

What is claimed is:

1. A transdermal patch comprising a plurality of discrete carbon nanotubes wherein a plurality of the discrete carbon nanotubes are open-ended at least at one end.

2. The transdermal patch of claim 1, wherein the plurality of discrete carbon nanotubes are dispersed within a medium fabricated into a fiber or film.

3. The transdermal patch of claim 1, wherein the plurality of discrete open-ended carbon nanotubes further comprise an amount of functional groups of at least about 1 percent by weight of the dry open-ended discrete carbon nanotubes.

4. The transdermal patch of claim 1, wherein the plurality of discrete open-ended carbon nanotubes are substantially cleaned of catalytic residues.

5. The transdermal patch of claim 1, wherein the plurality of discrete open-ended carbon nanotubes have a length less than about 4 micrometers, preferably less than about 3 micrometers and more preferably less than about 2 micrometers.

6. The transdermal patch of claim 1, wherein the plurality of discrete open-ended carbon nanotubes have a length distribution modality, preferably bimodal.

7. The transdermal patch of claim 1, wherein the plurality of discrete open-ended carbon nanotubes have a diameter distribution modality, preferably bimodal.

8. The transdermal patch of claim 1 wherein the plurality of discrete open-ended carbon nanotubes have an aspect ratio of from about 10 to about 500, preferably about 25 to about 200 and most preferably about 50 to about 120.

9. The transdermal patch of claim 1 further comprising a medicament.

10. The transdermal patch of claim 1, wherein the discrete carbon nanotubes are further associated with a polymer.

11. The transdermal patch of claim 10, wherein the polymer is selected from a group of polymers that do not exhibit substantial cytotoxity.

12. The transdermal patch of claim 10 wherein the polymer is selected from a group of polymers that are biodegradable.

13. The transdermal patch of claim 1, wherein the plurality of discrete open-ended carbon nanotubes are oriented in the direction that drug delivery is desired.

14. The transdermal patch of claim 1, comprising one or more layers wherein the plurality of discrete open-ended carbon nanotubes are oriented in the direction that drug delivery is desired.

15. The transdermal patch of claim 1, comprising one or more layers wherein discrete carbon nanotubes are in at least one layer to enable a lower voltage to be applied across the layer for the same rate of drug delivery as a transdermal patch without discrete carbon nanotubes.

16. The transdermal patch of claim 1, wherein at least a portion of discrete nanotubes preferably has a ratio of number average value of tube contour length (TCL)):tube end to end length (TEE) of from about 1.1 to about 3, preferably from about 1.1 to about 2.8, more preferably from about 1.1 to about 2.4, most preferably from about 1.1 to about 2 and especially form about 1.2 to about 2.

17. The transdermal patch of claim 1 wherein the polymer comprises a weight percentage range of about 1 to about 99, preferably less than about 90 percent, more preferably less than about 50 percent and most preferably less than about 25 percent of the discrete carbon nanotubes.

18. The transdermal patch of claim 1 wherein the discrete open-ended discrete carbon nanotubes comprises a weight percentage range of about 0.01 to about 20 percent, preferably less than about 10 percent, more preferably less than about 2 percent and most preferably less than about 1 percent of the material of the transdermal patch.

19. The transdermal patch of claim 1 wherein the discrete carbon nanotubes comprise a mixture of discrete carbon nanotubes with different types of attached functionalities.

20. The transdermal patch of claim 1, further comprising a pressure-sensitive adhesive material for adhering said transdermal patch to the skin or mucosa of a host.

21. A method to make a transdermal patch comprising a plurality of discrete carbon nanotubes comprising the steps of:
a) selecting a medium,
b) dispersing a plurality of discrete carbon nanotubes within the medium,
c) fabricating the polymer and the plurality of discrete carbon nanotube into a fiber or film,
d) optionally melt fabricating the film or fiber, and
e) optionally using one or more multilayer coextrusion generators.

22. The method of claim 20, wherein the medium is a polymer.

23. A method to make an active material layer for a transdermal patch comprising a plurality of discrete carbon nanotubes comprising the steps of:
a) selecting a medium,
b) selecting a medicament,
c) dispersing a plurality of discrete carbon nanotubes within the medium and medicament,
d) fabricating the medium, medicament and discrete carbon nanotube into a fiber or film,
e) optionally melt fabricating the film or fiber, and
f) optionally using one or more multilayer coextrusion generators.

24. The method of claim 21 further comprising joining one or more layers of medium.

25. A method to make a transdermal patch comprising a plurality of discrete open-ended carbon nanotubes wherein the discrete open-ended carbon nanotubes are deposited as a layer or on a layer surface by one or more deposition methods comprising spraying, inkjet printing, transfer printing, lamination, painting or spin coating.

26. The method of claim 23 further comprising joining one or more layers of mediums.

27. The transdermal patch of claim 9 further comprising a medicament and discrete open-ended carbon nanotubes in a reservoir.

28. The transdermal patch of claim 9 wherein a rate of delivery of the medicament is monitored by a change in dielectric constant across the patch.

29. The transdermal patch of claim 9 wherein a rate of delivery of the medicament is monitored by a change in resistance across the patch.

30. The transdermal patch of claim 9 wherein a rate of delivery of the medicament is controlled by an amount of infra-red, radio or microwave radiation.

31. A method of controlling rate of medicament ion transport with an applied voltage across a cathode and anode electrode, the method comprising
a) mixing a plurality of discrete carbon nanotubes in an aqueous mixture comprising medicament in an ionic form, wherein the plurality of discrete carbon nanotubes are mixed in an amount sufficient to increase conductivity of a film made from the mixture from at least 50% to 500% over the conductivity of a film consisting essentially of the same about amount of a plurality of discrete carbon nanotubes and an absence of the medicament,
b) placing the mixture between a cathode and anode electrode,
c) and applying voltage across the cathode electrode and electrode.

32. The method of claim 31 wherein the plurality of discrete carbon nanotubes are mixed in an amount from 0.01 weight percent to about 1 weight percent of the mixture.

33. A conductive transdermal patch layer comprising a medicament in ionic form and from about 0.01 to about 1 weight % of a plurality of discrete carbon nanotubes, wherein the patch has a conductivity of at least 50% greater than, and up to 500% greater than, a transdermal patch consisting essentially of the same about amount of a plurality of discrete carbon nanotubes and an absence of the medicament in ionic form.

34. The conductive transdermal patch of claim 33 comprising a medicament in ionic form and from about 0.25 to about 0.75% weight of a plurality of discrete carbon nanotubes, wherein the patch has a conductivity of at least 50% greater than, and up to 200% greater than, a transdermal patch consisting essentially of the same about amount of a plurality of discrete carbon nanotubes and an absence of the medicament.
